(19) United States
(12) Patent Application Publication
(10) Pub. No.: US 2026/0364243 A1
(43) Pub. Date: Nov. 19, 2026

(54) AIRWAY CLEARANCE DEVICE

(71) Applicant: University of Idaho, Moscow, ID (US)

(72) Inventor: Tao Xing, Moscow, ID (US)

(21) Appl. No.: 19/680,834

(22) Filed: May 18, 2026

Related U.S. Application Data
(60) Provisional application No. 63/808,322, filed on May 19, 2025.

(51) Int. Cl. A61M 1/00

(52) U.S. Cl. CPC A61M 1/774; A61M 1/69; A61M 1/741; A61M 1/79; A61M 2202/0007; A61M 2202/0014; A61M 2202/02; A61M 2202/06; A61M 2205/18; A61M 2205/3327; A61M 2205/3331; A61M 2205/50; A61M 2205/75; A61M 2210/1025

(57) ABSTRACT

An airway clearance device includes a first actuation member, a second actuation member, and a filtration member comprising first and second pipes defining an annular chamber. The first pipe includes a plurality of openings extending through a wall of the first pipe such that the openings are fluidly coupled to the annular chamber. The second pipe comprises a wall defining an inner lumen. The inner lumen is fluidly coupled to the first actuation member via a one-way valve such that actuation of the first actuation member is configured to provide a fluid to a subject’s lungs via the inner lumen. The annular chamber is fluidly coupled to the second actuation member via another one-way valve such that actuation of the second actuation member creates low or vacuum pressure configured to suck airway secretions from the subject’s airway into the annular chamber.

### CROSS-REFERENCE TO RELATED APPLICATION

This application claims the benefit of U.S. Provisional Patent Application No. 63/808,322, filed May. 19, 2025, which is incorporated by reference herein in its entirety.

### FIELD

The present application relates to a portable, economical, and effective airway clearance device configured to provide air to and remove airway secretions from a subject’s lungs.

### BACKGROUND

Airway secretions are common in chronic lung diseases, such as Chronic Obstructive Pulmonary Disease (COPD), cystic fibrosis, bronchiectasis, and asthma. For example, COPD, which is often associated with excess mucus, affects 11.7 million adults in the U.S. (5% of the adult population), and results in 3.5 million deaths per year worldwide. Mucus plugs occluding the airways of people with COPD are significantly associated with a higher risk of all-cause mortality. Asthma affects 26.8 million people in the U.S. (21.8 million adults and 5 million children).

Existing devices lack the ability to both deliver atmospheric/fresh air to the lungs and remove airway secretions (e.g., mucus) simultaneously and further can be costly, time-consuming, and uncomfortable for patients. Manual chest physiotherapy (CPT) is time-consuming, dependent on caregiver skill, and may be uncomfortable for patients. High-frequency chest wall oscillation is not portable, has an extremely high cost, and requires patients to sit for a 20-30 minute session. Positive expiratory pressure (PEP) devices, such as a flutter valve, may be difficult for weak and/or elderly patients to use. Intrapulmonary Percussive Ventilation (IPV) requires a trained operator, is not portable, and can be costly for regular use. Nebulized therapies may help thin mucus, but do not prevent buildup or clear the airway. Mechanical insufflation-exsufflation (alternating positive/negative pressure to the lungs) is not effective for thick and/or adherent mucus, such as is found in many conditions. Bronchoscopy and suctioning are invasive and require sedation and can further cause low oxygen levels (hypoxia).

Accordingly, a need exists for improved airway clearance devices that are easy to use, economical, and effective for providing air to and removing airway secretions (e.g., mucus) from a subject’s lungs/airway.

### SUMMARY

The present disclosure is directed to an airway clearance device configured to simultaneously, sequentially, or asynchronously deliver fluid to and remove secretions from a subject’s lungs/airway in a controlled manner. The device can include a filtration member sized and configured for placement within the airway, along with first and second actuation members fluidly coupled to the filtration member via separate flow paths. The first actuation member can be configured to introduce fluid, such as ambient or medicated air or oxygen, into the airway through a first flow path, while the second actuation member can be configured to extract secretions from the airway through a second, distinct flow path. In this manner, the device can provide coordinated, simultaneous, or asynchronous delivery and removal functions to assist in airway clearance and respiratory support.

In some examples, the filtration member can include a double-pipe configuration having an inner pipe defining an inner lumen for fluid delivery and an outer pipe defining, together with the inner pipe, an annular chamber for extracting airway secretions, such as mucus. A plurality of openings formed in the outer pipe can place the annular chamber in fluid communication with the airway to facilitate removal of secretions. The actuation members can include one-way valve arrangements to direct flow, such that actuation of the first actuation member drives fluid into the airway, while actuation of the second actuation member draws secretions out of the airway. Flexible connecting tubes can fluidly couple the actuation members to the filtration member. The actuation members can be configured to operate sequentially, simultaneously, substantially simultaneously, or asynchronously to enhance airway clearance efficiency.

Airway clearance devices described herein enable simultaneous airflow and mucus clearance in the same airway by facilitating a non-interfering counterflow of fluid delivery and mucus removal, ensuring that fluid (e.g., atmospheric/fresh air and/or medicated air) reaches a subject’s lungs while airway secretions are effectively removed and expelled therefrom. Further, the airway clearance devices described herein are portable and low-cost, making them more accessible than many or all current devices.

In some examples, an airway clearance device can comprise a filtration member configured to be arranged within a subject’s airway. In addition to this component, an airway clearance device can further comprise one or more of the features and/or components disclosed herein.

In some examples, the filtration member comprises a first pipe and a second pipe.

In some examples, the airway clearance device can comprise a first actuation member fluidly coupled to the filtration member via the second pipe defining a first flow path.

In some examples, the airway clearance device can comprise a second actuation member fluidly coupled to the filtration member via a chamber at least partially defined between the first pipe and the second pipe, the chamber defining a second flow path separate from the first flow path.

In some examples, the filtration member can be configured to deliver fluid into the subject’s airway via the first flow path when the first actuation member is actuated.

In some examples, the filtration member can be configured to extract secretions from the subject’s airway via the second flow path when the second actuation member is actuated.

In some examples, the second actuation member can be configured to collect the extracted secretions.

In some examples, the first and second actuation members can be bulbs.

In some examples, the first actuation member can comprise a first one-way valve and a second one-way valve.

In some examples, when the first actuation member is actuated, the second one-way valve can allow the fluid to be pushed from the first actuation member to the filtration member and the first one-way valve can allow the fluid to be drawn into the first actuation member.

In some examples, the second actuation member can comprise a third one-way valve and a fourth one-way valve.

In some examples, when the second actuation member is actuated, the third one-way valve can allow airway secretions to be drawn into the second actuation member from the filtration member and the fourth one-way valve can allow airway secretions to be expelled from the second actuation member.

In some examples, the first and second actuation members can be configured to be actuated simultaneously or substantially simultaneously.

In some examples, the first and second actuation members can be configured to be actuated sequentially.

In some examples, the first and second actuation members can be configured to be actuated asynchronously.

In some examples, the first pipe can comprise a first wall and a plurality of openings extending through the first wall.

In some examples, the second pipe can be disposed within the first pipe and can comprise a second wall forming an inner lumen.

In some examples, the first and second pipes can define an annular chamber disposed between the first and second walls and fluidly coupled to the plurality of openings.

In some examples, the inner lumen of the second pipe can define the first flow path configured to deliver a fluid to the subject’s airway when the filtration member is disposed within the subject’s airway.

In some examples, the annular chamber can define the second flow path configured to remove secretions from the subject’s airway.

In some examples, the airway clearance device can comprise a first flexible connecting tube fluidly coupling the first actuation member to the filtration member.

In some examples, a one-way valve can be disposed at a junction of the first connecting tube and the first actuation member.

In some examples, the airway clearance device can comprise a second flexible connecting tube fluidly coupling the second actuation member to the filtration member.

In some examples, a one-way valve can be disposed at a junction of the second connecting tube and the second actuation member.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an airway clearance device, according to an example, comprising a filtration member, a first actuation member, and a second actuation member.

FIG. 2 is a cross-sectional view of a distal end portion of the filtration member of the airway clearance device of FIG. 1.

FIG. 3 is a cross-sectional view of the distal end portion of the filtration member of the airway clearance device of FIG. 1 disposed within a subject’s airway.

FIG. 4 is a cross-sectional view of a proximal end portion of the filtration member of the airway clearance device of FIG. 1.

FIG. 5 is a cross-sectional view of the distal end portion of the filtration member of the airway clearance device taken along the line 5-5 in FIG. 2.

FIG. 6 is a perspective view of the distal end portion of the filtration member of FIG. 2.

FIG. 7 is a perspective view of the distal end portion of the filtration member of FIG. 2 comprising a diverging nozzle.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

In the description, certain terms may be used such as “forward,” “front,” “rear,” “back,” “up,” “down,” “upper,” “lower,” “horizontal,” “vertical,” “left,” “right,” “longitudinal,” “lateral,” and the like. These terms are used, where applicable, to provide some clarity of description when dealing with relative relationships. However, these terms are not intended to imply absolute relationships, positions, and/or orientations. For example, with respect to an object, an “upper” surface can become a “lower” surface by turning the object over. Nevertheless, it is still the same object.

As used herein, the term “distal” refers to a position, direction, or portion of a device that is further away from the user/physician and closer to the subject/lungs. Thus, for example, proximal motion of a device is motion of the device away from the lungs and toward the user (for example, out of the subject’s body), while distal motion of the device is motion of the device away from the user and toward the lungs (for example, into the subject’s body). The term “axial” refers to an axis extending in the proximal and distal directions, unless otherwise expressly defined.

As used herein, “e.g.” means “for example,” and “i.e.” means “that is.”

As used herein, values modified by the term “substantially” mean ± 10% of the stated value. For example, “substantially perpendicular” means an angle of 80° to 100° relative to a reference. “Substantially parallel” means an angle of ± 10° relative to a reference. “Substantially simultaneous” means occurring at the same time or within 2 seconds of one another. “Asynchronous” as used herein refers to actuation that is not simultaneous or substantially simultaneous. For example, actuating a first actuation member and actuating a second member 3 or more seconds after the first. Asynchronous can be a general term that includes sequential actuation and/or overlapping actuation.

### Overview of the Disclosed Technology

The airway clearance devices disclosed herein can establish separate, non-interfering flow paths for fluid delivery and secretion (e.g., mucus) removal within a subject’s airway. By separating these flow paths, the device can permit introduction of a fluid stream into the airway while concurrently or selectively withdrawing secretions. Such a configuration can enhance the effectiveness of mucus mobilization and evacuation while maintaining adequate airflow to the airway and lungs during operation. Maintaining airflow to the airway and lungs can help preserve airway patency, promote ventilation and oxygenation, and minimize the likelihood of airway collapse. Moreover, the airway clearance devices disclosed herein may advantageously provide improved safety relative to other suctioning devices by permitting simultaneous air delivery and mucus removal, thereby reducing transient interruptions in ventilation.

In some examples, a filtration member can be configured to support this dual-flow operation by providing physically distinct passages for inflow and outflow. The filtration member can comprise a counterflow, double-pipe configuration that can be arranged within a subject’s airway. For example, the counterflow double-pipe configuration can comprise an inner lumen through which fluid is delivered and a surrounding annular region or chamber through which airway secretions are removed. In some examples, the inner lumen may have a fluid outlet comprising a converging nozzle through which fluid exits the device for delivery into the subject’s airway. The converging nozzle outlet can be configured to boost velocity of the fluid that is delivered from the device. The fluid can enter the airway as a localized flow directed generally along a central region of the airway. In some examples, the nozzle may be a diverging nozzle.

Although mucus is described herein as one example of airway secretions, it is appreciated that any airway secretion may be removed via the airway clearance devices disclosed herein. Accordingly, the terms “mucus” and “secretions” are used interchangeably herein.

In some examples, the flow of fluid delivered may impart shear forces to mucus within the airway and may displace, loosen, mobilize, or redistribute the mucus from a more central location toward one or more sidewall regions of the airway. Mucus moved toward the sidewall regions of the airway may then be more readily removed, such as by suction, coughing, aspiration, or a combination thereof. Openings formed along a portion of the filtration member can enable communication between the annular chamber and the airway, allowing the mucus to be drawn into the device. The actuation members can be operated to generate pressure differentials within their respective flow paths, thereby promoting controlled delivery of fluid and removal of mucus, either sequentially, simultaneously, or substantially simultaneously.

The devices described herein thus promote optimal oxygen levels while effectively clearing lung airway secretions. Additionally, the airway clearance devices described herein can be portable, low-cost, and user-friendly, making them accessible and easily operable by a variety of users.

### Examples of the Disclosed Technologies

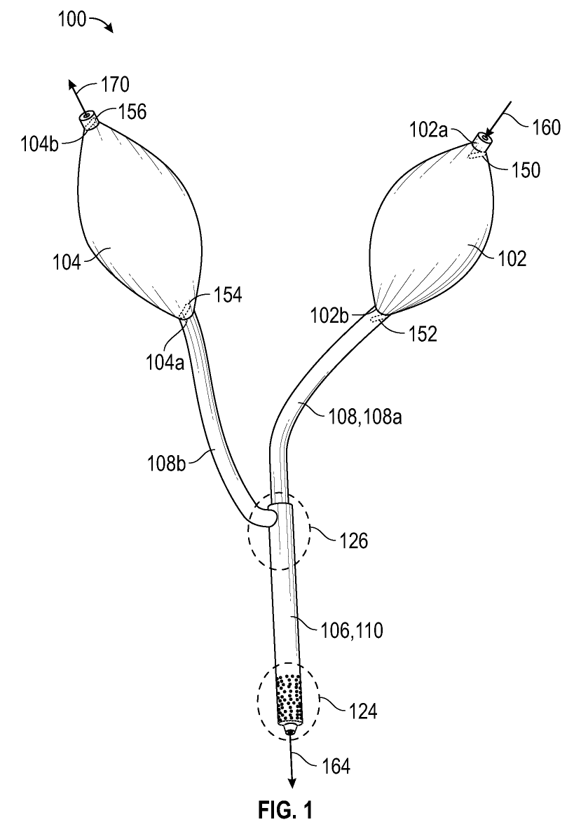

An exemplary airway clearance device 100 is illustrated in FIG. 1, according to an example. The airway clearance device 100 comprises a first actuation member 102, a second actuation member 104, and a filtration member 106 configured to be inserted into a subject’s airway 200 to deliver fluid (e.g., ambient air, oxygen, and/or medicated air and/or oxygen) into the subject’s lungs while removing airway secretions. The first and second actuation members 102, 104 can be fluidly coupled to the filtration member via two or more connecting tubes 108 (e.g., first connecting tube 108aand second connecting tube 108b). That is, the first actuation member 102 can be fluidly coupled to the filtration member 106 via the first connecting tube 108a and the second actuation member 104 can be fluidly coupled to the filtration member 106 via the second connecting tube 108b. In some examples, such as the illustrated example, the connecting tubes 108 may be flexible connecting tubes allowing a user to manipulate the position of the connecting tubes 108 and thereby the position of the actuation members 102, 104. Although the airway clearance device 100 is shown and described as having the first and second connecting tubes 108a, 108b, it is appreciated that the first and second connecting tubes 108a, 108b may be omitted as will be described further below. That is, the first and second actuation members 102, 104 can be directly connected to the filtration member 106, for example, via connectors, fittings, or junctions.

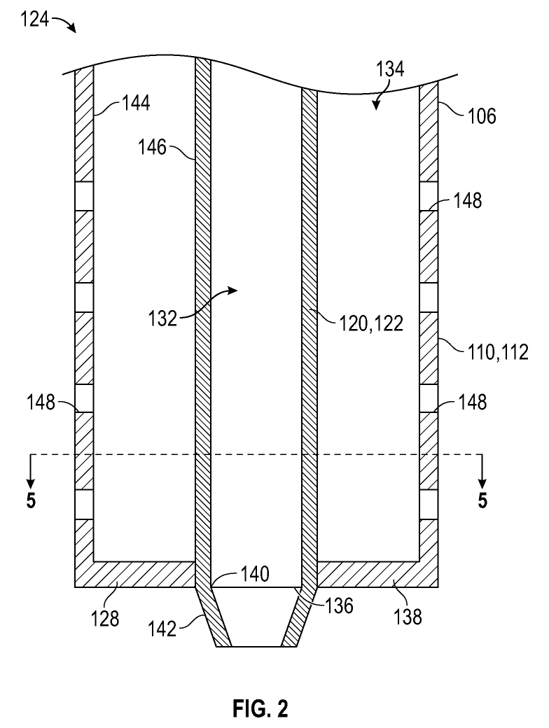

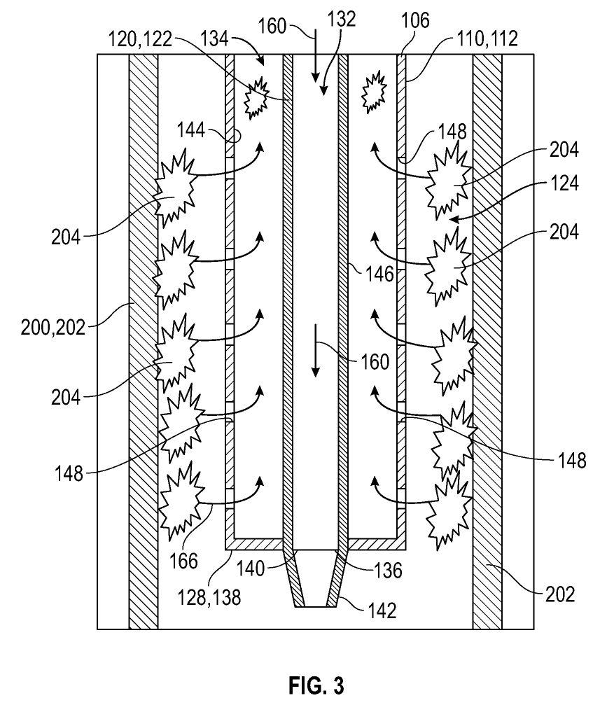

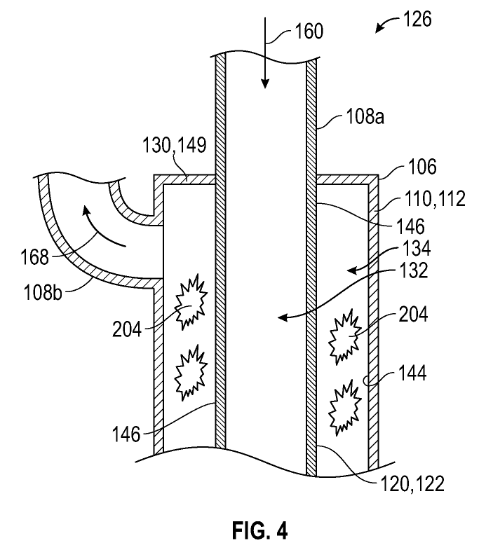

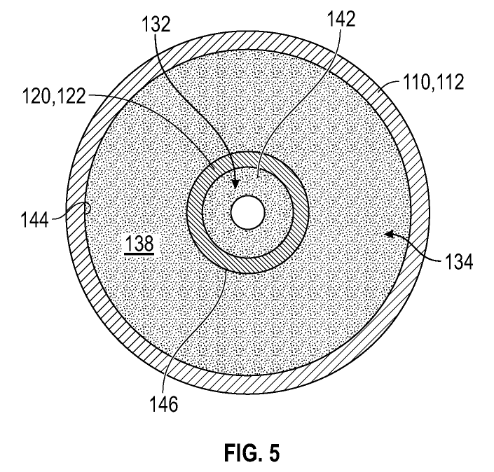

As shown in FIGS. 2-3, the filtration member 106 can have a counterflow, double-pipe configuration comprising an outer pipe 110 (also referred to herein as a “first pipe”) having a first wall 112 and an inner pipe 120 (also referred to herein as a “second pipe”) having a second wall 122 disposed within the outer pipe 110. In some examples, the first wall 112 of the outer pipe 110 and the second wall 122 of the inner pipe 120 can each be substantially cylindrical in shape, thereby defining generally concentric or coaxial flow passages as shown in FIG. 5. FIG. 2 illustrates a cross-sectional view of a distal end portion 124 of the filtration member 106 and FIG. 3 illustrates a cross-sectional view of the distal end portion 124 of the filtration member 106 inserted within walls 202 of a subject’s airway 200. FIG. 4 illustrates a cross-sectional view of a proximal end portion 126 of the filtration member 106 coupled to the first and second connecting tubes 108a, 108b. Although described herein as pipes, the outer and inner pipes 110, 120 can be cylindrical members, shafts, tubes, conduits, or the like.

The filtration member 106 comprising the outer and inner pipes 110, 120 can accordingly have a generally cylindrical configuration comprising a distal end 128 (FIGS. 2-3) and a proximal end 130 (FIG. 4). An outer profile of the filtration member 106 can be sized to be easily inserted into the airway 200.

The outer and inner pipes 110, 120 forming the filtration member 106 can comprise a flexible material selected to improve maneuverability and facilitate insertion into the subject’s airway. In some examples, the outer and inner pipes 110, 120 can be formed from or comprise biocompatible materials such as medical-grade silicone, polyurethane, polyvinyl chloride (PVC), or other elastomeric and/or polymeric materials that provide sufficient flexibility, durability, and resistance to kinking. In some examples, one or more portions of the filtration member 106 can alternatively or additionally be formed from or comprise metals (e.g., stainless steel, titanium, or aluminum) or combinations of metallic and polymeric materials, which can provide increased structural integrity. In some examples, at least the filtration member 106 can be made of materials configured to withstand repeated sterilization processes (e.g., autoclaving or chemical sterilization). This can advantageously allow the device 100 to be sterilized and reused.

As shown in FIGS. 2-3, the inner pipe 120 can be disposed within the outer pipe 110 (e.g., coaxially) such that the second wall 122 defines an inner lumen 132 for fluid delivery. The space between the first wall 112 and the second wall 122 defines an annular passage or chamber 134 as shown in FIG. 5. Such cylindrical configurations can facilitate improved patient comfort, reduced flow resistance, and simplified manufacturing, although other cross-sectional shapes (e.g., oval, rectangular, or the like) are also possible. Although the outer and inner pipes 110, 120 are shown and described herein as being coaxial or substantially coaxial, it is appreciated that longitudinal axes of the outer and inner pipes 110, 120 may be offset from one another. In some examples, the first wall 112 and the second wall 122 may touch one another such that the space between the first wall 112 and the second wall 122 is crescent-shaped.

The inner lumen 132 of the inner pipe 120 extends from the proximal end 130 of the filtration member 106 (see e.g., FIG. 4) to the distal end 128 of the filtration member 106 (see e.g., FIGS. 2-3) and has a distal end 136 that is substantially aligned with a distal end 138 of the outer pipe 110. In other words, the distal ends 136, 138 of the inner and outer pipes 120, 110, respectively, define the distal end 128 of the filtration member 106. The inner pipe 120 comprises an outlet 140 at its distal end 136 through which the fluid can be delivered from the inner lumen 132 when the filtration member 106 is arranged within the airway 200. In some examples, the outlet 140 of the inner pipe 120 can comprise a nozzle 142, for example a converging nozzle. The converging nozzle 142 positioned at the outlet 140 of the inner pipe 120 can increase the velocity of fluid delivered to the airway, which can assist in breaking up mucus 204 (FIG. 3), for example, in the airway, pushing mucus closer to the airway walls 202. Breaking up the mucus and/or pushing the mucus closer to the airway walls 202 can help to preserve airway patency, promote ventilation and oxygenation, minimize the likelihood of airway collapse, and improve the safety of mucus clearance.

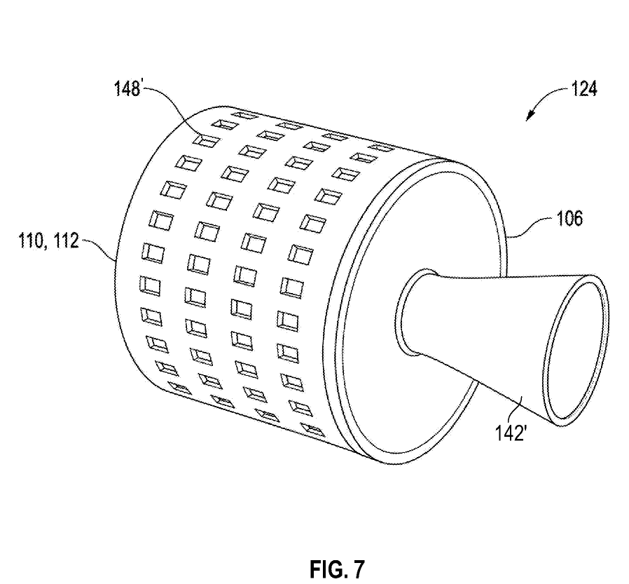

In some implementations, the nozzle can be a diverging nozzle 142´ as shown in FIG. 7 and described further below.

As shown in FIG. 4, the inner lumen 132 can be fluidly connected to the first connecting tube 108a at the proximal end 130 of the filtration member 106.

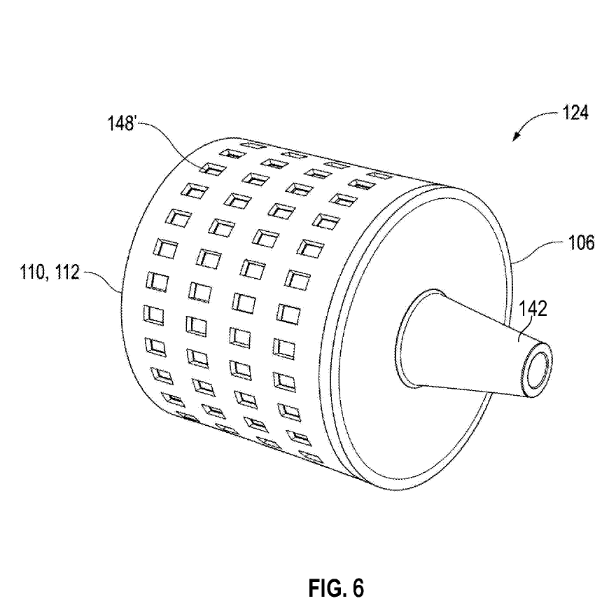

As shown in FIGS. 2-4, the annular chamber 134 is bound by an inner surface 144 of the wall 112, an outer surface 146 of the wall 122, the distal end 128 of the filtration member 106, and the proximal end 130 of the filtration member 106. As shown in FIG. 1, the distal end portion 124 of the filtration member 106 may comprise a plurality of openings 148 extending through a thickness of the wall 112 of the outer pipe 110. While FIG. 1 illustrates a plurality of openings 148 having circular shapes, it should be understood that the openings can have any of various shapes. For example, FIGS. 6-7 illustrate implementations of the distal end portion of the outer pipe 110 comprising openings 148´ each having a rectangular shape. Further, while FIG. 1 illustrates twelve rows of openings, it should be understood that, in other implementations, the outer pipe 110 can comprise a lesser or greater number of rows. For example, FIGS. 6-7 illustrate implementations comprising four rows of openings, and FIG. 3 illustrates an implementation comprising five rows of openings. Additionally, while some implementations, such as FIGS. 2-3 and 6-7, illustrate the rows of openings being circumferentially aligned with one another, in other implementations, the openings in one row may be circumferentially offset from the openings in one or more adjacent rows.

The plurality of openings 148 are thus in fluid communication with the annular chamber 134 and fluidly couple the annular chamber 134 to the surrounding airway 200 as shown in FIG. 3. FIG. 3 illustrates an implementation wherein the distal end portion of the outer pipe 110 comprises 5 rows of openings 148. In some examples, the openings 148 can include 2-10 rows of openings, 3-8 rows of openings, or 5 rows of openings spaced apart from one another axially along the distal end portion of the outer pipe 110. FIG. 6 illustrates an implementation wherein the distal end portion of the outer pipe 110 comprises 4 rows of openings 148´. Such an implementation can be used in lieu of or in addition to the implementation shown in FIG. 1. Each row of openings can comprise a plurality of openings spaced apart from one another about a circumference of the outer pipe 110. In other examples, the outer pipe 110 can comprise a greater or fewer number of rows and/or openings, for example, including a different number, size, shape, and location of the openings, depending on the application. For example, the openings 148 may have any suitable shape, including but not limited to square, rectangular, triangular, circular, oval, elongated, slotted, or irregular shapes. As shown for example in FIG. 6, in some implementations, the openings 148´ may have a rectangular shape.

In some examples, the openings 148 can extend along the length of the entire outer pipe 110 from the distal end 128 to the proximal end 130 of the filtration member 106.

Apart from the openings 148, as shown in FIGS. 2-4, the annular chamber 134 can be enclosed at its first, distal end by the distal end 138 of the outer pipe 110 and at its second, proximal end 130 by an end wall 149. As will be described further below, when the second actuation member 104 is actuated, mucus (e.g., represented by shapes 204 in FIGS. 3-4) from the subject’s lungs and/or airway can be sucked through the openings 148 in the outer pipe 110 and into the annular chamber 134 formed between the inner and outer pipes 120, 110. That is, the annular chamber 134 defined by the outer surface 146 of the inner pipe 120 and an inner surface 144 of the outer pipe 110 receives mucus that is extracted from the subject’s airway. The annular chamber 134 can be coupled to the second connecting tube 108b at a proximal end portion 126 of the filtration member 106 as shown in FIG. 4. Accordingly, mucus can be received within the annular chamber 134 via the openings 148 and collected therefrom by the second actuation member 104.

As shown in FIG. 4, the first connecting tube 108a can couple the first actuation member 102 to the inner pipe 120 of the filtration member 106 (e.g., the inner lumen 132), and the second connecting tube 108b can couple the second actuation member 104 to the outer pipe 110 of the filtration member 106 (e.g., the annular chamber 134). The first and second actuation members 102, 104 can comprise, for example, bulbs (e.g., rubber bulb syringes) as depicted in FIG. 1. Although bulbs are primarily shown and described herein, it is appreciated that the airway clearance device 100 may comprise suitable alternative actuation structures that can provide pulsed fluid flow via actuation (e.g., bellows, pistons, spring-biased chambers, motor-driven pumps, or the like). For example, the first actuation member 102 may comprise a mechanical ventilator, in some examples, and/or the second actuation member 104 may comprise a mucus suction machine.

Although the first connecting tube 108a is shown and described herein as a continuous tube extending from the actuation member 102 to the filtration member 106, it is appreciated that the first connecting tube 108a may be configured to additionally couple with other devices. For example, a nebulizer can be fluidly coupled to the first connecting tube 108a through an intermediate connector, such as a T-junction, a Y-junction, or another multi-port junction. By coupling to a nebulizer, medication from the nebulizer can be introduced into the airflow delivered by actuation of the actuation member 102. The nebulizer may be configured to deliver an aerosolized agent, including a mucolytic, saline, bronchodilator, or other therapeutic formulation, to thin, loosen, or break up mucus within the airway, thereby facilitating mucus clearance by coughing, suctioning, or both.

Turning again to FIG. 1, the first actuation member 102 can be configured as a fluid (e.g., air) supplier, such as a bulb as described above, having a first end portion 102a and a second end portion 102b. The first end portion 102a can comprise a first one-way valve 150 (also referred to as a “check valve”). In examples in which the first actuation member 102 draws ambient, atmospheric air from the outside environment, the first one-way valve 150 regulates the passage of the ambient, atmospheric air from the outside environment into the inner volume of the first actuation member 102. That is, the first one-way valve 150 provides an interface between the inner volume of the first actuation member 102 and the outside environment.

In examples in which the first actuation member 102 is coupled to a container or other source (e.g., containing air/oxygen, or other substances to be delivered to the lungs, such as medication), the first one-way valve 150 may be disposed between the inner volume of the first actuation member 102 and the source. Accordingly, the first one-way valve 150 can be configured to allow a fluid from the source, such as medicated air/oxygen, to enter the first actuation member 102.

The second end portion 102b of the first actuation member 102 can comprise a second one-way valve 152 disposed at the junction between the first actuation member 102 and the first connecting tube 108a. The second one-way valve 152 can be configured to allow the fluid to move from the first actuation member 102 into the inner pipe 120 (see FIGS. 2-4) of the filtration member 106 (e.g., into the inner lumen 132).

In some examples, the second one-way valve 152 can be disposed within any portion of the first connecting tube 108a.

In other examples, the second one-way valve 152 can be coupled directly to the inner pipe 120 of the filtration member 106. That is, in some examples, the first connecting tube 108a may be omitted from the device 100. In such examples, the second one-way valve 152 and/or the first actuation member 102 can be directly coupled to the filtration member 106 via a connector, fitting, junction, or the like.

The second actuation member 104 can be configured as a mucus collector having a first end portion 104a and a second end portion 104b. The first end portion 104acan comprise a third one-way valve 154 disposed between the second connecting tube 108b and the second actuation member 104. That is, the third one-way valve 154 can be arranged at the first end portion 104a of the second actuation member 104 and can be configured to allow mucus to flow from the filtration member 106 via the second connecting tube 108b into the second actuation member 104. In some examples, the third one-way valve 154 is disposed at the junction of the second connecting tube 108b and the second actuation member 104.

In some examples, the third one-way valve 154 can be disposed within any portion of the second connecting tube 108b.

In other examples, the third one-way valve 154 can be coupled directly to the outer pipe 110 of the filtration member 106. That is, in some examples, the second connecting tube 108b may be omitted from the device 100. In such examples, the third one-way valve 154 and/or the second actuation member 104 can be directly coupled to the filtration member 106 via a connector, fitting, junction, or the like.

Mucus that collects in the second actuation member 104 can be emptied from the inner volume of the second actuation member 104 via a fourth one-way valve 156 located at the second end portion 104bof the second actuation member 104. For example, the fourth one-way valve 156 can separate the inner volume of the second actuation member 104 from the outside environment. In other words, the fourth one-way valve 156 provides an interface between the inner volume of the second actuation member 104 and the outside environment (e.g., a bag, container, or other mucus catching device). The fourth one-way valve 156 can be configured to allow mucus to be expelled or removed from the second actuation member 104. As will be described further below, by squeezing the second actuation member 104, mucus contained within the second actuation member 104 can be expelled through the fourth one-way valve 156 while the third one-way valve 154 inhibits backflow toward the filtration member 106.

In operation, the second end portion 102b of the first actuation member 102 can be fluidly coupled to the filtration member 106 (e.g., through the first connecting tube 108a) such that actuation (e.g., compression and/or expansion, for example, manual compression, squeezing, or manipulation) of the first actuation member 102 acts to expel fluid (e.g., air) from the first actuation member 102 and into the inner pipe 120 (e.g., the inner lumen 132) of the filtration member 106, as depicted by arrows 160 in FIGS. 3-4. For example, a user can actuate the first actuation member 102 (e.g., by manually compressing/squeezing the member) to increase the pressure inside the actuation member 102. As a result of the actuation, the first valve 150 closes and the second valve 152 opens. The first one-way valve 150 inhibits backflow from the first actuation member 102 through the first end portion 102a and into the environment (or container). Similarly, the second one-way valve 152 inhibits backflow from the first connecting tube 108a and/or the inner pipe 120 into the first actuation member 102.

The increased pressure created inside the first actuation member 102 when actuated drives the fluid stored in the first actuation member 102 to enter the inner pipe 120 (e.g., via the connecting tube 108a) for delivery to the lungs, for example, through the outlet 140. When the user releases the first actuation member 102, the air pressure in the actuation member 102 decreases, causing the second valve 152 to close and the first valve 150 to open. That is, upon release, the first actuation member 102 can resiliently return to its original or undeformed shape, drawing fluid (e.g., air from the environment or a container), as depicted by arrow 160 in FIG. 1, into the inner volume of the actuation member 102 through the first one-way valve 150, recharging the first actuation member 102 for subsequent actuation. By repeating this action, the fluid can be continuously pumped into the subject’s lungs via the inner pipe 120, as depicted by arrow 164 in FIG. 1.

Similarly, the first end portion 104a of the second actuation member 104 can be fluidly coupled to the filtration member 106 (e.g., through the second connecting tube 108b) such that actuation (e.g., manual compression and release) of the second actuation member 104 acts to suck mucus from the filtration member 106 (e.g., the annular chamber 134) into the second actuation member 104 and expel collected mucus from the second actuation member 104. More specifically, when the user squeezes then releases the second actuation member 104, the actuation member 104 can resiliently return to its original shape upon release. Expansion of the actuation member can create a reduced pressure (e.g., a partial vacuum) within the inner volume of the second actuation member 104 relative to the pressure within the second connecting tube 108b, thereby facilitating suction of mucus from the filtration member 106 into the second actuation member 104 through the third one-way valve 154. That is, the reduced pressure in the actuation member 104 causes the fourth valve 156 to close and the third valve 154 to open. As a result, low and/or vacuum pressure created in the second actuation member 104 sucks mucus through the openings 148 in the filtration member 106, into the annular chamber 134 between the inner and outer pipes 120, 110, as depicted by arrows 166 in FIG. 3, and into the second actuation member 104, as depicted by arrow 168 in FIG. 4 (e.g., through the second connecting tube 108b).

When the user actuates the second actuation member 104 (e.g., via compression/squeezing), pressure inside the actuation member 104 increases. As a result of this increased pressure, the third valve 154 closes and the fourth valve 156 opens. The increased pressure created inside the second actuation member 104 expels mucus stored therein, as depicted by arrow 170 in FIG. 1, for example, into a sink or a container (e.g., a collection bag or the like). By repeating actuation of the second bulb 104, mucus can be continuously sucked into the second actuation member 104 and expelled therefrom.

In some examples, actuation of the first and second actuation members 102, 104 (e.g., squeezing and releasing) can be performed simultaneously or substantially simultaneously.

In some examples, actuation of the first and second actuation members 102, 104 (e.g., squeezing and releasing) can be performed sequentially, for example, alternating actuation of the first and second actuation members 102, 104, depending on the needs of the patient. For example, if additional mucus removal is desired, the second actuation member 104 may be actuated more frequently. Conversely, if additional fluid delivery is desired, the first actuation member 102 may be actuated more frequently. Actuation of the first and second actuation members 102, 104 may be advantageously implemented as required or desired.

In some examples, actuation of the first and second actuation members 102, 104 (e.g., squeezing and releasing) can be performed asynchronously such that the actuation of the members 102, 104 is not simultaneous or substantially simultaneous. For example, actuation of the first and second actuation members 102, 104 can be performed in an overlapping manner and/or sequentially.

It is also appreciated that either of the actuation members 102, 104 may be implemented without the other actuation member. For example, if only fluid delivery is desired, the actuation member 102 may be actuated in the absence of the actuation member 104. Conversely, if only mucus removal is desired, the actuation member 104 may be actuated in the absence of the actuation member 102.

The disclosed device advantageously provides separate flow streams or channels for fluid (e.g., via lumen 132 of inner pipe 120) and mucus (e.g., via annular chamber 134 between the outer and inner pipes 110, 120), which avoids interference between the two streams. This separation significantly increases the effectiveness and efficacy of the mucus removal while maintaining a healthy oxygen level and positive pressure in the subject’s lungs.

In some examples, the airway clearance devices disclosed herein may further comprise one or more sensing and control features configured to monitor and improve safety and performance during clearance and ventilation. For example, one or more pressure sensors, flow meters, oxygen sensors, carbon dioxide sensors, temperature sensors, humidity sensors, and/or mucus-property sensors may be embedded within, mounted on, integrated with, or otherwise coupled to any portion of an airway clearance device. For example, sensors may be arranged on or within any one or more tubes (e.g., tubes 108a, 108b), actuation members (e.g., the first and second actuation members 102, 104), and/or portions of a filtration member (e.g., the outer and inner pipes 110, 120) to measure local pressure, flow rate, oxygen level, carbon dioxide level, temperature, humidity, mucus presence, mucus thickness, mucus viscosity, mucus concentration, or combinations thereof. The measurements or data from the one or more sensors may be used to detect unsafe conditions, such as excessive positive pressure, excessive suction, blockage, elevated airway resistance, inadequate ventilation, abnormal gas levels, undesirable temperature or humidity conditions, changes in mucus characteristics, or the like. The measurements may trigger a warning, alarm, or other response.

In some examples, a controller may receive and analyze sensor data from the one or more sensors using threshold-based logic, feedback control, machine-learning methods, artificial-intelligence methods, or combinations thereof, and may provide guidance to an operator to adjust one or more operating parameters, including fresh air delivery, suction level, tidal volume, pulse frequency, peak pressure, gas conditioning, or other therapy settings, to improve mucus removal, maintain adequate ventilation, and enhance patient safety. Such operating parameters may further include one or more gas-delivery parameters, suction parameters, pressure parameters, flow parameters, timing parameters, pulse parameters, cycling parameters, gas-composition parameters, and alarm parameters, including but not limited to delivered pressure, baseline pressure, mean pressure, flow rate, delivered volume, cycling frequency, inspiratory timing, expiratory timing, pulse amplitude, pulse duration, oxygen concentration, humidity level, temperature, sensitivity settings, support level, and threshold limits. In some examples, adjustment of one or more operating parameters may occur automatically or semi-automatically, for example, without user involvement or with minimal user involvement.

In some examples, the controller may adjust operating parameters in response to measured or derived conditions including excessive or insufficient pressure, excessive suction, elevated flow resistance, abnormal gas composition, inadequate gas exchange, blockage, secretion burden, gas trapping, or other unsafe or undesirable operating conditions, thereby enabling safe operation in ventilation modes, mucus-clearance modes, or combined ventilation and mucus-clearance modes.

In some examples, the relative geometry, opening arrangement, and functional assignment of the outer and inner pipes (e.g., outer and inner pipes 110, 120) may be varied while retaining the combined mucus-clearance and ventilation functionality of the disclosed device. For example, in certain embodiments, the outer pipe 110 may omit one or more side-wall openings otherwise provided along a distal portion thereof, such that the outer pipe 110 delivers fluid substantially through an open distal end. In such configurations, the inner pipe 120 may be extended farther distally, proximally, or both distally and proximally, relative to the outer pipe. Extending the inner pipe 120 in this manner may increase separation between a fresh-air discharge region and a suction region, thereby reducing interference between supplied airflow and aspirated mucus and/or improving mucus-removal effectiveness.

In other examples, the functional roles of the inner pipe and the outer pipe (e.g., outer and inner pipes 110, 120) may be varied (or reversed) while retaining the combined mucus-clearance and ventilation functionality. For example, the pipe (e.g., inner pipe 120) previously used for fresh air delivery may instead be configured for suction and mucus removal, and the pipe (e.g., outer pipe 110) previously used for suction and mucus removal may instead be configured to deliver fresh air. In such an arrangement, the nozzle can be configured as the diverging nozzle 142´ shown in FIG. 7 to more effectively entrain mucus. More generally, either pipe may serve as an air-supply passage, a suction passage, or a combined therapeutic-flow passage. Accordingly, the lengths, relative axial positions, terminal openings, side openings, and spacing of the pipes may be configured to achieve a desired balance of ventilation, mucus mobilization, secretion capture, flow separation, and patient safety.

In some examples, the suction pipe, the fresh-air supply pipe, or both the suction and fresh-air supply pipes (e.g., outer and inner pipes 110, 120) may be configured to rotate, spin, oscillate, index, reciprocate circumferentially, or otherwise move about a longitudinal axis or another axis relative to one another and/or relative to a surrounding support structure in order to enhance device performance. Such motion may be continuous, intermittent, pulsatile, stepwise, periodic, aperiodic, synchronized with suction, synchronized with air delivery, or independently controlled. Rotation or other motion of the suction pipe may improve mucus-removal effectiveness by varying the circumferential position of one or more suction openings (e.g., openings 148, 148´), minimizing the risk of localized clogging, distributing suction over multiple regions, increasing local shear or flow disturbance near mucus deposits, and/or promoting swirling flow, turbulence, or other enhanced flow mixing that facilitates mucus dislodgement, entrainment, and transport.

Rotation or other motion of the fresh-air supply pipe may impart a tangential component to the delivered airflow, thereby producing a swirling, helical, turbulent, or otherwise non-axisymmetric flow that may enhance mixing, flow distribution, mucus mobilization, ventilation effectiveness, suction effectiveness, or combinations thereof.

In some examples, such rotational or other motion may be produced manually or by one or more drive arrangements, including but not limited to a motor, actuator, gear arrangement, belt drive, friction drive, magnetic coupling, fluid-driven element, turbine-driven element, pneumatic drive, hydraulic drive, electromechanical drive, compliant mechanism, linkage, transmission, bearing-supported assembly, or combinations thereof. In some examples, the rotational speed, angular position, direction of rotation, oscillation amplitude, duty cycle, timing, synchronization, and other motion characteristics may be selectively adjusted or controlled to achieve a desired balance of mucus clearance, airflow delivery, secretion capture, turbulence generation, reduced blockage, enhanced mixing, and patient safety.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An airway clearance device comprising:
a filtration member configured to be arranged within a subject’s airway and comprising a first pipe and a second pipe;
a first actuation member fluidly coupled to the filtration member via the second pipe defining a first flow path; and
a second actuation member fluidly coupled to the filtration member via a chamber at least partially defined between the first pipe and the second pipe, the chamber defining a second flow path separate from the first flow path,
wherein the filtration member is configured to deliver fluid into the subject’s airway via the first flow path when the first actuation member is actuated, and
wherein the filtration member is configured to extract airway secretions from the subject’s airway via the second flow path when the second actuation member is actuated.

2. The airway clearance device of claim 1, wherein the second actuation member is configured to collect the extracted airway secretions.

3. The airway clearance device of claim 1, wherein the first actuation member comprises a first one-way valve and a second one-way valve, and wherein when the first actuation member is actuated, the second one-way valve allows the fluid to be pushed from the first actuation member to the filtration member and the first one-way valve allows the fluid to be drawn into the first actuation member.

4. The airway clearance device of claim 1, wherein the second pipe is disposed within the first pipe.

5. The airway clearance device of claim 1, wherein the second actuation member comprises a third one-way valve and a fourth one-way valve, and wherein when the second actuation member is actuated, the third one-way valve allows airway secretions to be drawn into the second actuation member from the filtration member and the fourth one-way valve allows airway secretions to be expelled from the second actuation member.

6. The airway clearance device of claim 1, wherein the first and second actuation members are configured to be actuated simultaneously or substantially simultaneously.

7. An airway clearance device comprising:
a filtration member configured to be positioned within a subject’s airway, the filtration member comprising:
a first pipe comprising a first wall and a plurality of openings extending through the first wall; and
a second pipe disposed within the first pipe and comprising a second wall forming an inner lumen,
wherein the first and second pipes define an annular chamber disposed between the first and second walls and fluidly coupled to the plurality of openings,
wherein the inner lumen of the second pipe defines a first flow path configured to deliver a fluid to the subject’s airway when the filtration member is disposed within the subject’s airway, and
wherein the annular chamber defines a second flow path configured to remove airway secretions from the subject’s airway.

8. The airway clearance device of claim 7, further comprising a first actuation member fluidly coupled to the inner lumen of the second pipe.

9. The airway clearance device of claim 8, wherein the first actuation member comprises a one-way valve configured to allow fluid into the first actuation member.

10. The airway clearance device of claim 7, further comprising a second actuation member fluidly coupled to the annular chamber.

11. The airway clearance device of claim 10, wherein the second actuation member comprises a one-way valve configured to allow airway secretions to be drawn into the second actuation member.

12. The airway clearance device of claim 8, further comprising a first flexible connecting tube fluidly coupling the first actuation member to the filtration member, wherein a one-way valve is disposed at a junction of the first flexible connecting tube and the first actuation member.

13. The airway clearance device of claim 10, further comprising a second flexible connecting tube fluidly coupling the second actuation member to the filtration member, wherein a one-way valve is disposed at a junction of the second flexible connecting tube and the second actuation member.

14. An airway clearance device comprising:
a filtration member configured to be disposed within a subject’s airway, the filtration member comprising:
a first pipe comprising a first wall and a plurality of openings extending through the first wall; and
a second pipe disposed within the first pipe and comprising a second wall defining an inner lumen,
wherein the first and second pipes define an annular chamber between the first wall and the second wall, and
wherein the annular chamber is fluidly coupled to the plurality of openings;
a first actuation member fluidly coupled to the inner lumen and, when actuated, configured to deliver a fluid to the subject’s airway via the inner lumen; and
a second actuation member fluidly coupled to the annular chamber and, when actuated, configured to draw airway secretions from the subject’s airway through the plurality of openings and into the second actuation member via the annular chamber.

15. The airway clearance device of claim 14, wherein the fluid comprises atmospheric air or medicated air.

16. The airway clearance device of claim 14, wherein the inner lumen of the second pipe comprises an outlet configured to direct the fluid to the subject’s airway, the outlet comprising a nozzle.

17. The airway clearance device of claim 14, wherein the plurality of openings are disposed on a distal end portion of the first pipe.

18. The airway clearance device of claim 14, further comprising a first tube extending from the first actuation member to a proximal end portion of the inner lumen, wherein a first one-way valve is disposed between the first tube and the first actuation member.

19. The airway clearance device of claim 14, further comprising a second tube extending from the second actuation member to a proximal end portion of the annular chamber, wherein a second one-way valve is disposed between the second tube and the second actuation member.

20. The airway clearance device of claim 14, further comprising:
one or more sensors configured to measure one or more operating parameters; and
a controller configured to receive one or more measurements from the one or more sensors,
wherein, based on at least the one or more measurements, the controller is configured to generate an alert, adjust one or more operating parameters of the device, or generate an alert and adjust one or more operating parameters of the device.
